SEQUENCE LISTING (4 sequences)

SEQ ID NO: 1 - protein, 14 aa, Homo sapiens
KKFKNSTYSRSSVD

SEQ ID NO: 2 - protein, 16 aa, Homo sapiens
KLYGPDVGQPRRKTAK

SEQ ID NO: 3 - protein, 22 aa, Homo sapiens
KKLVGTKVLMASVQGSKRRKLR

SEQ ID NO: 4 - protein, 15 aa, Homo sapiens
KKFKNSTYSRSSVDC

(19) United States
(12) Patent Application Publication
(10) Pub. No.: US 2011/0104140 A1
(43) Pub. Date: May 5, 2011

(54) USE OF NON-CATALYTIC FORM OF HEPARANASE AND PEPTIDES THEREOF FOR REVERSING THE ANTI-COAGULANT EFFECTS OF HEPARINOIDS

(76) Inventors: Israel Vlodavsky, Mevasseret Zion (IL); Neta Ilan, Rehovot (IL); Flonia Levy-Adam, Haifa (IL); Ben-Zion Katz, Rehovot (IL)

(21) Appl. No.: 12/299,985

(22) PCT Filed: May 10, 2007
(86) PCT No.: PCT/IL2007/000564

§ 371 Date: Aug. 6, 2009

(30) Foreign Application Priority Data
May 11, 2006 (IL) 175571

(51) Int. Cl. A61K 38/47; A61K 38/36; A61P 7/02; C12Q 1/56

(52) U.S. Cl. 424/94.61; 514/13.7; 435/13

(57) ABSTRACT

The present invention relates to inhibition of heparinoids anti-coagulation activity by a non-active form of a eukaryotic endoglycosidase or any fragment or peptide thereof comprising at least one heparin-binding domain. More particularly, the invention provides compositions and methods for the inhibition of heparinoids anti-coagulation activity and for the treatment of coagulation related pathologic clinical conditions, using a non-active form of mammalian heparanase or peptides thereof comprising at least one heparin-binding domain.

### FIELD OF THE INVENTION

The present invention relates to methods and compositions for the treatment of coagulation related pathologic clinical conditions. More particularly, the invention provides the use of a non-active form of mammalian heparanase or peptides thereof for inhibiting heparinoids anti-coagulation activity and thereby treating coagulation related pathologic clinical conditions.

### BACKGROUND OF THE INVENTION

All publications mentioned throughout this application are fully incorporated herein by reference, including all references cited therein.

One of the major physiological roles of the endothelium is to preserve the integrity of the vasculature by its permeability barrier properties, and to provide a non-thrombogenic surface. Therefore, the endothelial cell surface serves as a prime regulatory site of coagulation responses. Under normal circumstances, an injury to vascular endothelial cells lining a blood vessel triggers a hemostatic response through a sequence of events commonly referred to as the “coagulation cascade”. The cascade culminates in the conversion of soluble fibrinogen to insoluble fibrin which, together with platelets, forms a localized clot or thrombus which prevents extravasation of blood components. Wound healing can then occur followed by clot dissolution and restoration of blood vessel integrity and flow.

The events which occur between injury and clot formation are carefully regulated and linked series of reactions, involving number of plasma coagulation proteins in inactive proenzyme forms and cofactors circulate in the blood. Active enzyme complexes are assembled at an injury site and are sequentially activated to serine proteases, with each successive serine protease catalyzing the subsequent proenzyme to protease activation. This enzymatic cascade results in each step magnifying the effect of the succeeding step. In brief, clot formation is mediated by the conversion of fibrinogen to fibrin by thrombin. Thrombin is generated by the prothrombinase complex that includes Factor Xa, Factor Va and prothrombin on endothelial cells, as well as fibroblasts, haematopoietic cells surfaces. Various regulatory mechanisms operate to restrict clot formation to conditions required by physiologically regulated hemostasis, e.g. blood vessel injury. A key element for this physiological restriction of clotting is the non-thrombogenic properties of the endothelium surface. Following the initiation by a tissue factor-dependent mechanism, initial minor quantities of thrombin induce a positive feedback amplification of the intrinsic coagulation pathway aiming to generate sufficient quantities of thrombin for the conversion of fibrinogen to fibrin. Moreover, thrombin is generated from prothrombin by the prothrombinase complex (containing factors Xa and Va) on the surface of disturbed endothelial cells, as well as on fibroblasts and haematopoietic cells [Autin L. et al. Proteins 63:440-450 (2006)].

Anti-coagulant properties of cell-surfaces have been previously attributed to heparan sulfate proteoglycans (HSPG). HSPG are macromolecules composed of a core protein covalently O-linked to repeating hexuronic and D-glucosamine disaccharide units containing sulfated side chains that have been shown to exert anti-coagulant activities in cells, extracellular matrix (ECM) and tissues. Cell surface and clinically administrated HSPG molecules have been previously shown to associate with components of the coagulation system, including Factor Xa and the natural thrombin inhibitor antithrombin III (ATIII).

Moreover, cell surface HSPG can facilitate the catabolism of coagulation factors such as FVIII [Sarafanov A. G. et al. J. Biol. Chem. 276:11970-11979 (2001)]. Other coagulation inhibitors such as tissue-factor-pathway-inhibitor also associate with the external face of endothelial cell plasma membrane via HSPG [Ho G. et al. J. Biol. Chem. 272:16838-16844 (1997)]. HSPG are also important constituents of the sub-endothelial basement membrane, where they cross-link various components, e.g. laminin, collagens, thereby contributing to the integrity of the blood vessel wall [Iozzo R. V. Nat. Rev. Mol. Cell Biol. 6:646-656 (2005)]. The initiation of coagulation response on the endothelium, or in the subendothelial basement membrane will require the impedance of the anticoagulant activities of HSPG. The only mammalian enzyme identified so far with specific heparan sulfate degrading activity is the endo-β-D-glucuronidase heparanase that is further discussed in more detail hereinafter.

While efficient clotting limits the loss of blood at an injury site, inappropriate formation of thrombi in veins or arteries is a common cause of disability and death. Abnormal clotting activity can result in and/or from pathologies or treatments such as myocardial infarction, unstable angina, atrial fibrillation, stroke, renal damage, percutaneous translumenal coronary angioplasty, disseminated intravascular coagulation, sepsis, pulmonary embolism and deep vein thrombosis. The formation of clots on foreign surfaces of artificial organs, shunts and prostheses such as artificial heart valves is also problematic.

Stroke is a leading cause of death and a common cause of permanent disability. The acute focal cerebral ischemia resulting in the neurological deficits of stroke are most frequently caused by thromboembolism. Thrombi can be generated from cardiac sources and atheromas. In situ thrombosis can occur in the large, extracerebral brain-supplying vessels. Studies suggest a finite time interval after cerebral arterial occlusion beyond which significant irreversible neuronal damage and sustained neurological deficit occurs.

Approved anticoagulant agents currently used in treatment of these pathologies and other thrombotic and embolic disorders include the sulfated heteropolysaccharides heparin and low molecular weight heparin (LMWH). These agents are administered parenterally and can cause rapid and complete inhibition of clotting.

Heparin is a linear polysaccharide produced by mast cells and composed of a polymer of alternating derivatives of D-glucosamine (N-sulfated or N-acetylated) and uronic acid (L-iduronic or D-glucuronic acid) linked by glycosidic linkages [Casu B. and Lindahl U., Adv. Carbohydr. Chem. Biochem. 57: 159-206 (2001); Robinson H C. et al. J. Biol. Chem. 253: 6687-93 (1978)]. Heparin is structurally related to heparin sulfate (HS), but has higher N- and O-sulfate contents [Casu B. and Lindahl U. (2001) ibid.]. The main anticoagulant effect of heparin has been attributed to its ability to catalyze the inhibitory reaction between AT and its target proteases: thrombin (Factor IIa) and factor Xa (FXa) [Bourin M C. and Lindahl U., Biochem. 289: 313-30 (1993)], while the main effect of low-molecular-weight heparin (LMWH) is because of AT-mediated inhibition of FXa activity [Hemker H. C. and Beguin S. Haemostasis 20: 81-92 (1990)]. As indicated above, heparin and LMWH are commonly used as anticoagulants in a range of diseases. Their levels are monitored indirectly by parameters inferred from blood samples drawn from patients on a regular basis. These parameters include activated partial thromboplastin time (APTT), thrombin time, and anti-Xa activity (for monitoring LMWH). These tests evaluate the coagulation profile of different factors in the coagulation cascade and the inhibitory effect of heparin and LMWH through activation of AT.

However, due to their potency, heparin and LMWH suffer drawbacks. Uncontrolled bleeding as a result of the simple stresses of motion and accompanying contacts with physical objects or at surgical sites is the major complication and is observed in 1 to 7% of patients receiving continuous infusion and in 8 to 14% of patients given intermittent bolus doses. To minimize this risk, samples are continuously drawn to enable ex vivo clotting times to be continuously monitored, which contributes substantially to the cost of therapy and the patient's inconvenience. Moreover, approximately 5% (range up to 30%) of patients treated with heparin develop immune-mediated thrombocytopenia (HIT) which may be complicated by either bleeding (as a consequence of decreased platelet count) or arterial and venous thrombosis due to intravascular platelet clumping. This complication occurs in as many as 20% of patients with HIT and may result in serious morbidity and death in about 50% of the cases. Therefore currently, a strong need exist to provide substances and methods for rapidly and efficiently reversing the anti-coagulant effects of different heparinoids. More particularly, there is need for an anti-dot to inhibit these clinically highly abundant anti-coagulants, in the case of urgent clinical needs. Such needs may occur during extensive bleedings (e.g. brain) caused by LMWH overdose or un-controlled bleedings in LMWH treated individuals due to other medical causes.

As indicated above, mammalian endoglycosidase, capable of partially depolymerizing HS chains and commonly referred to as heparanase, has been identified in a variety of cell types and tissues, primarily cancer cells, activated cells of the immune system, platelets, and placenta [Parish C R et al. Biochim. Biophys. Acta. 1471: 0M99-108 (2001); Vlodaysky I. and Friedmann Y. J. Clin. Invest. 108: 341-7 (2001); Nakajima M. et al. J. Cell Biochem. 36: 157-67 (1988); Dempsey L A., et al. Trends Biochem. 25: 349-51 (2000)]. Interestingly, only a single heparanase cDNA sequence encoding an active enzyme was identified, indicating that this enzyme is the dominant endo-β-D-glucuronidase in mammalian tissue [Vlodaysky I., et al. Nat. Med. 5:793-802 (1999); Hulett M D, et al. Nat. Med. 5:803-9 (1999); Kussie P H., et al. Biochem. Biophys. Res. Commun. 261: 183-7 (1999); Toyoshima M. and Nakajima M., J. Biol. Chem. 274: 24153-60 (1999)]. While exerting a variety of biological activities including degradation of the sub-endothelial basement membrane, stimulation of neo-vascularization, promoting cell adhesion and regulating gene-expression [Vlodaysky I. et al. Semin. Cancer Biol. 12:121-129 (2002); Vlodaysky I. et al. Pathophysiol. Haemost. Thromb. 35:116-127 (2006)], the possible roles of heparanase in the regulation of coagulation responses have not been studied in detail. Heparanase is synthesized as a latent 65 kDa precursor whose activation involves proteolytic cleavage at two potential sites located at the N-terminal region of the molecule (Glu¹⁰⁹-Ser¹¹⁰ and Gln¹⁵⁷-lys¹⁵⁸), resulting in the formation of two protein subunits, 8 and 50 kDa polypeptides, that heterodimerize and form the active heparanase enzyme [McKenzie E. et al. Biochemical J. 373: 423-35 (2003); Levy-Adam F. et al. Biochem. Biophy. Res. Commun. 308: 885-91 (2003)]. One of the prime physiological sources for heparanase are platelets [Hulett M. D. et al. Nat. Med. 5:803-809 (1999); Freeman C. and Parish C. R. Biochem. J. 330:1341-1350 (1998)]. The 50 and 8 kDa heparanase polypeptides were biochemically purified from platelets, which also contain significant amounts of the 65 kDa proenzyme [Hulett (1999) ibid; Freeman (1998) ibid.]. Moreover, the heparanase gene was previously cloned from human platelets [Hulett (1999) ibid.]. Heparanase released by activated platelets or platelet-derived microparticles, is biologically active, stimulates angiogenesis and modulates endothelial cell activities [Brill A. et al. Cardiovasc. Res. 63:226-235 (2004); Myler H. A. and West J. L. J. Biochem. 131:913-922 (2002)].

Processing of macro-molecular heparin was demonstrated in cultured mast cells, mostly by heparanase activity [Jacobsson K. G. and Lindahl U. Biochm. J. 246: 409-15 (1987); Gong F. et al. J. Biol. Chem. 278: 35152-8 (2003)]. The resulting degradation products correspond to the size of commercial heparin [Gong (2003) ibid.].

Early studies with platelet heparanase showed that it could cleave the glucuronide linkage in oligosaccharides containing the antithrombin (AT)-binding sequence of heparin and that the cleavage products lacked affinity for AT [Ogren S. and Lindahl U., J. Biol. Chem. 250: 2690-7 (1975)]. The mastocytoma heparanase has been implicated with the intra-cellular postbiosynthetic modification of heparin [Gong (2003) ibid/.]. Because heparanse cleavage of macromolecular heparin in mast cells generated products that contain the AT-binding sequence, mast cell heparanase was assumed to differ from platelet heparanese [Ogren (1975) ibid.; Thunberg, L. et al. J. Biol. Chem. 257: 10278-82 (1982)]. This assumption was debated, however, when the same heparanase was cloned from platelets and mast cells [Gong (2003) ibid.]. The AT-binding region of macromolecular heparin was shown to escape degradation by heparanase, while the cleavage of oligosaccharides with high affinity to AT was quite inefficient [Gong (2003) ibid.; Pikas D S. et al. J. Biol. Chem. 273: 18770-7 (1998)]. Attempts to define the substrate specificity of heparanase pointed to the importance of sulfation, but have otherwise failed to provide a unified conclusion [Pikas (1998) ibid.; Okada Y. et al. J. Biol. Chem. 277: 42488-95 (2002)]. Both early [Jacobsson and Lindahl (1987) ibid.] and recent [Pikas (1998) ibid.; Okada (2002) ibid.] studies clearly pointed to the β-D-glucuronic linkages as the target of heparanase.

A recent study performed by some of the present inventors, demonstrated that the enzymatic activity of heparanase may partially inhibit the anticoagulant activities of heparinoids [Nasser N. J. et al. J. Thromb. Haemost. 4:560-565 (2006)]. More particularly, this study show that heparanase is capable of degrading heparin and LMWH, and thereby to suppress the anticoagulant activity of heparin and LMWH, as indicated by a decreased effect on APTT and anti-Xa activity, respectively, when human plasma was added.

The inventors therefore hypothesized that the enzymatic activity of heparanase may be responsible for inhibiting the anti-coagulant effect of heparin. However, such a mechanism depends on relatively prolonged incubation times (i.e. hours) of the active enzyme with heparinoids under acidic conditions (pH<6.0) optimal for heparanase enzymatic activity [Nasser (2006) ibid.]. In contrast, procoagulant physiological activity should be exerted within minutes, under normal physiological conditions (e.g. neutral pH), and hence may involve other modes of heparanase effects, independent of its enzymatic activities. Therefore, there is need for fast acting antidote for reversing heparinoids anti-coagulating activity, particularly under physiological conditions.

The inventors thus proposed recently, to apply these findings as an indirect approach to quantify heparanase activity by measuring the decrease in plasma APTT or anti-Xa activity exerted by the enzyme under the defined conditions [Nasser (2006) ibid].

Nevertheless, when the present inventors further examined the role of heparanase in coagulation modulation, they surprisingly and unexpectedly found, as shown by the present invention, that the non-active form of heparanase, the heparanase pro-enzyme, has profound inhibitory effects on heparinoids-mediated regulation of coagulation responses, via mechanisms that are not enzymatic, and most importantly, particularly under physiological conditions. The inventors show that heparanase proenzyme did not directly affect the coagulation protein activities, but the protein (as well as a specific peptide thereof) has profound effects on heparinoid-mediated regulation of coagulation proteases, apparently via mechanisms that do not involve heparanase enzymatic activity.

More particularly, heparanase pro-enzyme reverses the anti-coagulant activity of unfractionated heparin on the intrinsic coagulation pathway as well as on thrombin activity. In addition, heparanase pro-enzyme abrogated the factor X inhibitory activity of low molecular weight heparin. The pro-coagulant effects of the non-active heparanase were also exerted by a peptide comprising its major functional heparin-binding sequence. Finally, the effects of heparanase on the activity of factor VII activating protease that is auto-activated by heparinoids indicated a complete antagonistic action of heparanase in this system. Altogether, heparanase pro-coagulant activities that were also demonstrated in plasma samples from patients under low molecular weight heparin treatment, point to a possible use of this molecule as antidote for heparinoid therapies.

It is therefore one object of the invention to provide a composition for the inhibition of heparinoids anti-coagulating activity, comprising a non-active form of heparanase or any fragments and peptides thereof comprising the heparin-binding site.

In yet another object, the invention provides methods for the treatment of a subject suffering of a coagulation related pathologic clinical condition, using the non-active form of heparanase and peptides thereof, particularly the peptide of SEQ ID NO: 1.

Another object of the invention is to provide the use of the non-active form of heparanase and particularly, of peptide having the amino acid sequence as denoted by SEQ ID NO: 1, for the preparation of a pharmaceutical composition for the treatment of a coagulation related pathologic clinical condition.

These and other objects of the invention will become apparent as the description precedes.

### SUMMARY OF THE INVENTION

In a first aspect, the present invention relates to a composition for the inhibition of heparinoids anti-coagulation activity. The composition of the invention comprises as an active ingredient a eukaryotic endoglycosidase, preferably, a non-active endoglycosidase, more preferably, non-active form of heparanase or any mutant, fragment or peptide thereof comprising at least one heparin-binding domain. This composition may optionally further comprise a pharmaceutically acceptable carrier, diluent excipient and/or additive.

The invention further relates to a composition for the inhibition of heparinoids anti-coagulation activity in a subject in need thereof. Such composition comprises as an active ingredient an eukaryotic endoglycosidase or any mutant, fragment or peptide thereof comprising at least one heparin-binding domain, said composition optionally further comprising a pharmaceutically acceptable carrier, diluent excipient and/or additive.

The invention further provides a composition for the treatment and prevention of a coagulation related pathologic clinical condition. Such composition comprises as an active ingredient an eukaryotic endoglycosidase or any mutant, fragment or peptide thereof comprising at least one heparin-binding domain, said composition optionally further comprising a pharmaceutically acceptable carrier, diluent excipient and/or additive.

In a second aspect, the invention relates to a method for the treatment and/or prevention of a subject suffering from a coagulation-related pathologic clinical condition, comprising the step of administering to said subject an inhibitory effective amount of a eukaryotic endoglycosidase or any mutant, fragment or peptide thereof comprising at least one heparin-binding domain or of any composition comprising the same.

The present invention further provides for a method for the inhibition of heparinoids anti-coagulation activity. This method comprises the steps of: (a) contacting an inhibitory effective amount of a eukaryotic endoglycosidase or any mutant, fragment or peptide thereof comprising at least one heparin-binding domain or of any composition comprising the same, with said heparinoid under suitable conditions creating a mixture; (b) adding to the mixture obtained in step (a), a mammalian body fluid sample, preferably plasma, under suitable conditions for a suitable period of time; and (c) examining the anticoagulation activity of said heparinoids on said sample, as compared to a suitable control, by a suitable means.

According to a third aspect, the present invention relates to the use of a eukaryotic endoglycosidase or any mutant, fragment or peptide thereof comprising at least one heparin-binding domain, in the preparation of a composition for the inhibition of heparinoids anti-coagulation activity, preferably, in a subject in need thereof.

Still further, the invention relates to the use of a eukaryotic endoglycosidase or any mutant, fragment or peptide thereof comprising at least one heparin-binding domain, in the preparation of a composition for the inhibition of heparinoids anti-coagulation activity.

The principles and operation of the present invention may be better understood with reference to the drawings and accompanying descriptions.

### BRIEF DESCRIPTION OF THE INVENTION

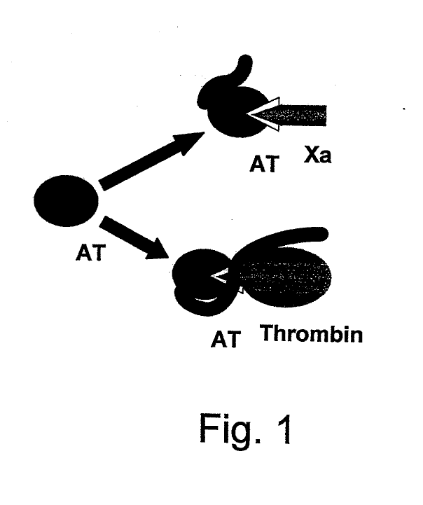

FIG. 1 Mechanism of Anti-Coagulant Activities of Heparinoids

Low molecular weight heparinoids (top) bind antithrombin (AT), and modulate its conformation enabling binding and inhibiting of active factor Xa. Unfractionated heparin (bottom) forms a ternary complex with AT and thrombin thereby inhibiting its enzymatic activity.

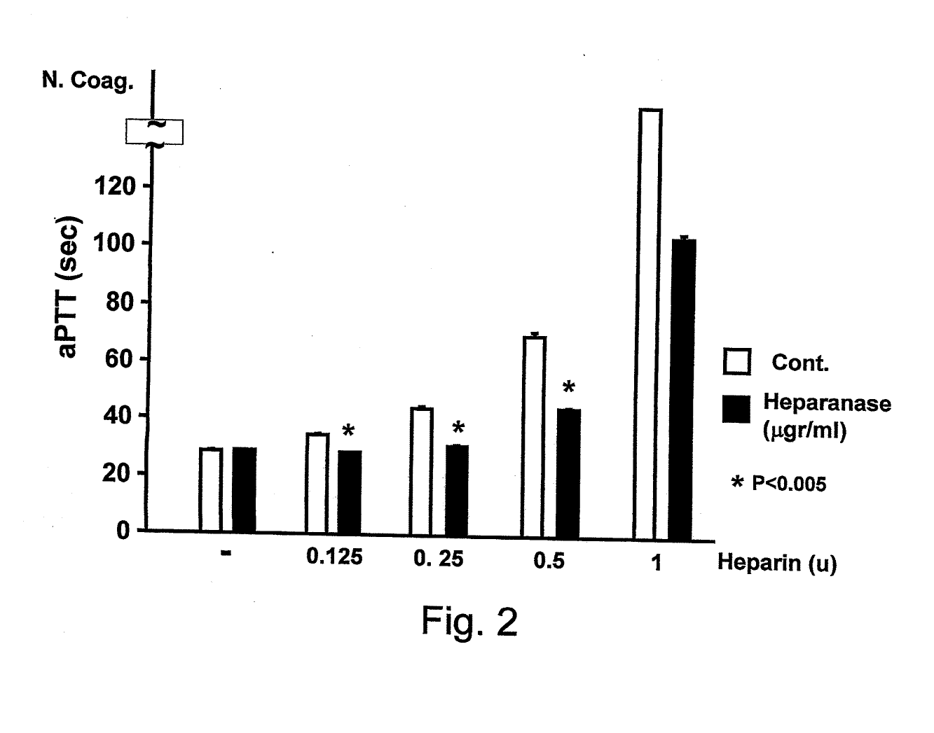

FIG. 2 Heparanase Pro-Enzyme Reverses the Heparin Anti-Coagulant Effects on the aPTT Response

In the aPTT assay, clot is generated within normal plasma by the intrinsic coagulation pathway. Clot formation is significantly inhibited by heparin, with complete coagulation inhibition in 1 u/ml heparin. The anticoagulant heparin effects are significantly reversed by heparanase pro-enzyme (one representative experiment out of four is shown). Abbreviations: cont. (control), N. coag. (No coagulation).

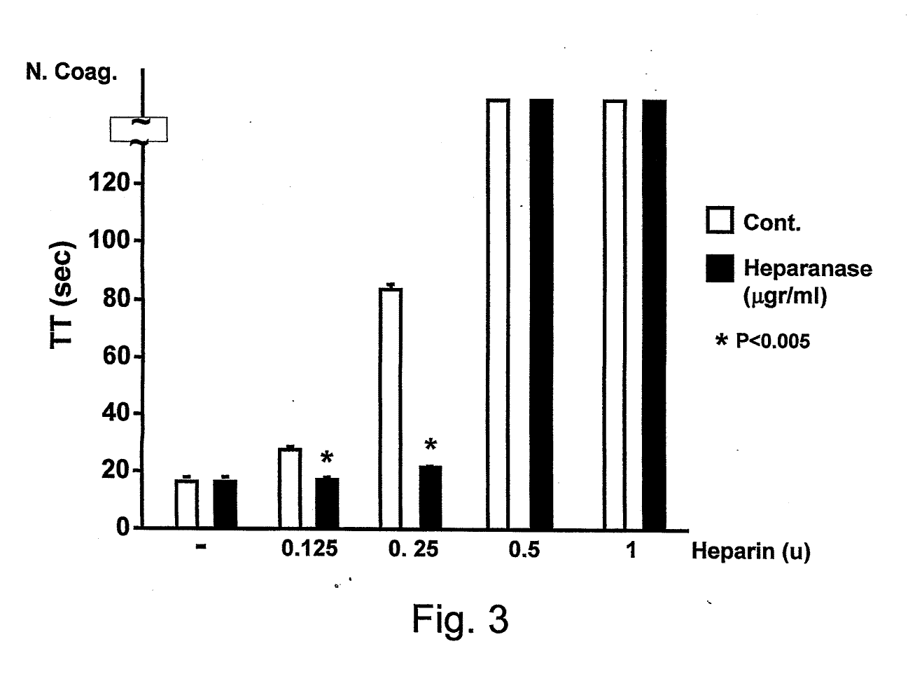

FIG. 3 Heparanase Pro-Enzyme Reverses the Heparin Anti-Coagulant Effects on Thrombin Activity

In the thrombin time assay (TTA), clot is generated within normal plasma. Clot formation is significantly inhibited by heparin, with complete coagulation inhibition in 0.5 u/ml heparin. The anticoagulant heparin effects are significantly reversed by heparanase pro-enzyme (one representative experiment out of three is shown). Abbreviations: cont. (control), sec. (seconds), N. coag. (No coagulation).

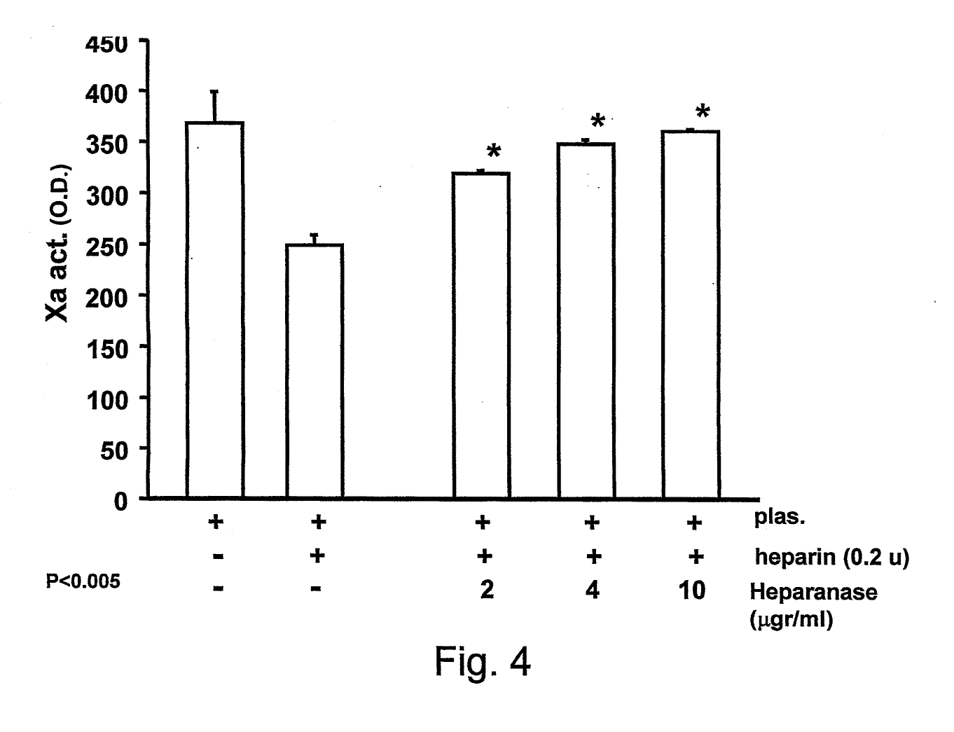

FIG. 4 Heparanase Pro-Enzyme Reverses the Heparin Anti-Xa Activity

Heparin inhibits the activity of activated coagulation factor X (Xa). Heparanase pro-enzyme reverses the anti-Xa activity of heparin, restoring Xa activity to normal levels. Abbreviations: act. (activity), plas. (plasma).

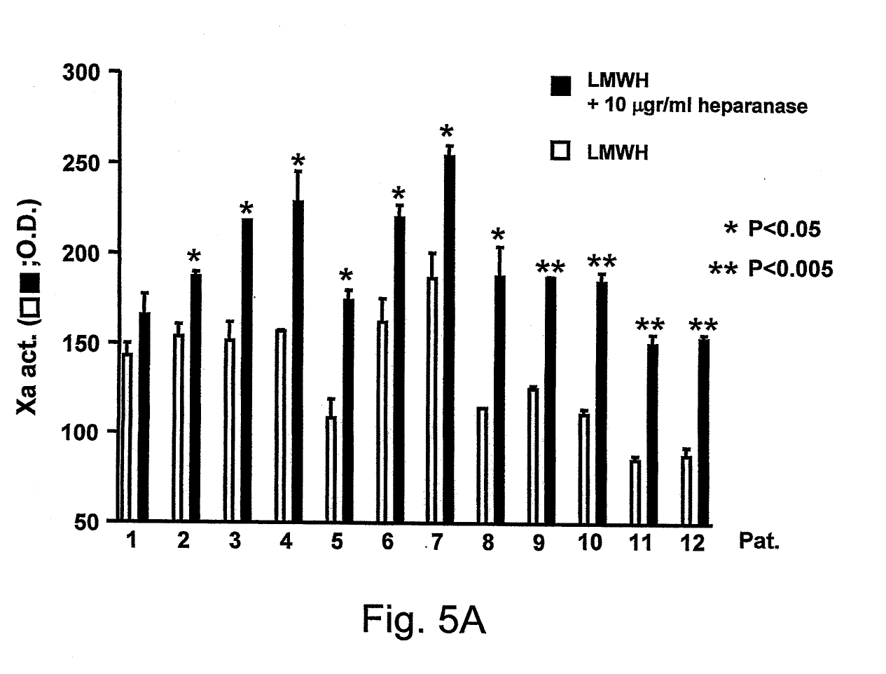

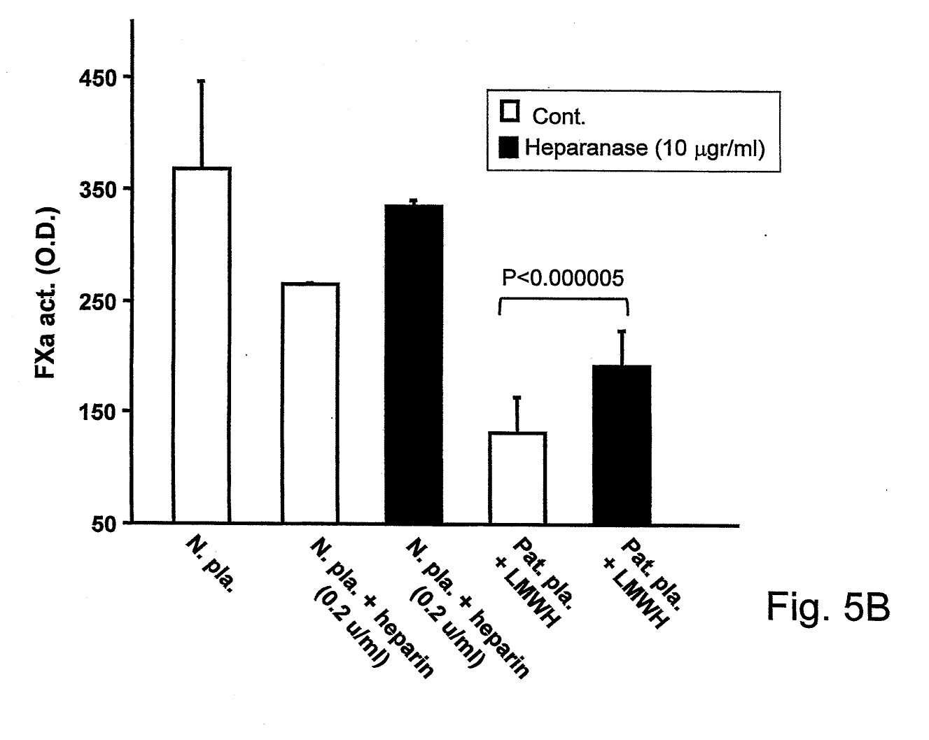

FIG. 5A-5B Heparanase Pro-Enzyme Reverses the LMWH Anti-Xa Activity in Clinical Samples

FIG. 5A. Heparanase pro-enzyme was added to plasma derived from twelve independent LMWH-treated patients. Factor Xa activity was then measured, within 5 min. Each sample was measured in the absence (open bars) or presence (filled bars) of μg/ml recombinant pro-heparanase. Each measurement was performed in duplicates (the average of each duplicate is shown, with differences between measurements <10%). A significant elevation of Factor Xa activity is evident (basal levels in LMWH-treated patients is ˜140 O.D.).

FIG. 5B. Summary of the effect of pro-heparanase on Factor Xa activity in the presence of heparin treated plasma, and the 12 LMWH-treated patient derived plasma samples (average±S.D.). Abbreviations: act. (activity), pat. (patient), pla. (plasma) cont. (control).

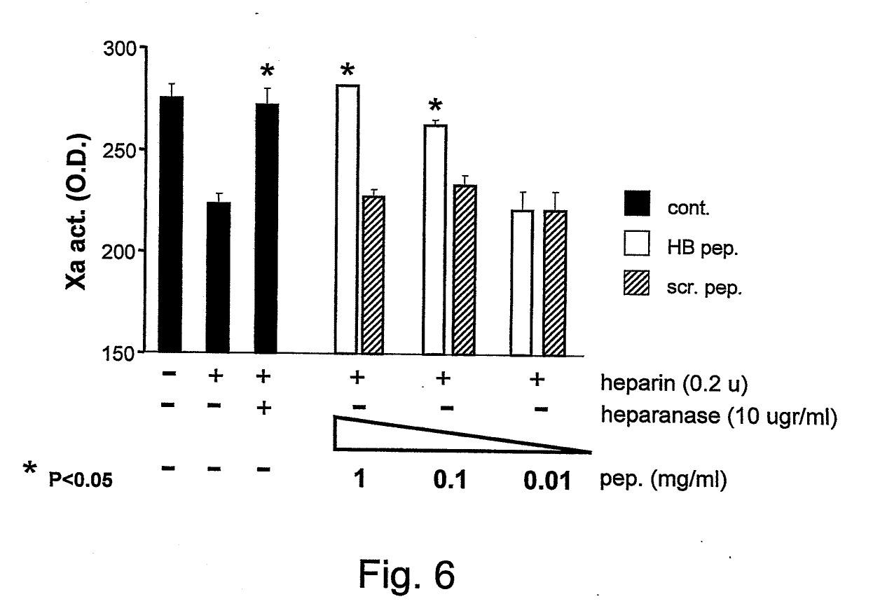

FIG. 6 The Lys158-Asp171 (Also Denoted by SEQ ID NO. 1) Heparin-Binding Peptide of Heparanase can Abolish the FXa Inhibitory Effects of Heparin

Heparin inhibits the activity of activated coagulation factor X (Xa). The Lys158-Asp171 heparin binding (HB) peptide of heparanase abolished the FXa inhibitory effects of heparin, in a dose dependent manner, thereby restoring Xa activity to normal levels. The control, scrambled peptide had no effect. Abbreviations: act. (activity), con. (control), scr. (scrambled), pep. (peptide).

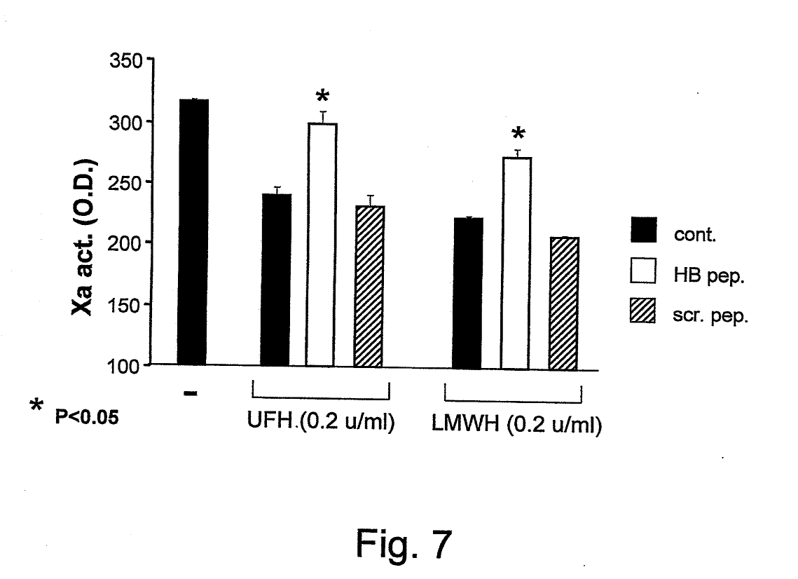

FIG. 7. The Lys158-Asp171 Heparin Binding Peptide of Heparanase can Abolish the FXa Inhibitory Effects of LMWH

Heparin and LMWH both inhibits the activity of activated coagulation factor X (Xa). The Lys158-Asp171 heparin binding (HB) peptide of heparanase abolished the FXa inhibitory effects of heparin and LMWH, thereby restoring Xa activity to normal levels. The control, scrambled peptide had no effect. Abbreviations: act. (activity), con. (control), scr. (scrambled), pep. (peptide).

### DETAILED DESCRIPTION OF THE INVENTION

Before explaining at least one embodiment of the invention in detail, it is to be understood that the invention is not limited in its application to the details set forth in the following description or illustrated in the drawings. The invention is capable of other embodiments or of being practiced or carried out in various ways. Also, it is to be understood that the phraseology and terminology employed herein is for the purpose of description and should not be regarded as limiting.

Hoemostasis and blood coagulation in particular are tightly regulated physiological processes that involve a series of molecular activation and inhibition events as well as the major contribution of different biological surfaces. Moreover, specific plasma proteins govern the rate of clot formation by control of coagulation serine proteases in conjunction with heparinoids as catalysts. Key regulators in this regard are AT and heparin-cofactor II that efficiently block thrombin and FXa in the presence of heparin or dermatan sulfate, respectively [Du H. Y, et al. Thromb. Res. 119:377-384 (2007); Taylor K. R. and Gallo R. L. FASEB J. 20:9-22 (2006)], whereby the AT-heparin system is of utmost clinical and therapeutic importance.

Several types of endothelial cells provide a non-thrombogenic surface, based on the expression of cell membrane-connected HS molecules that are able to bind basic proteins including AT, thereby inhibiting thrombin and possibly FXa activities [Labarrere C. A. et al. J. Heart Lung Transplant. 11:342-347 (1992)]. Other cell types (e.g. fibroblasts) also produce anti-coagulant heparinoids [Brandt J. T. et al. Thromb. Res. 51:187-196 (1988)]. Conversely, the balancing of HS-related reactions requires their neutralization or blockade, and such activities were previously attributed to basic proteins/peptides that are released from platelets upon activation. In particular, platelet factor 4 has been demonstrated to significantly prevent or inhibit the anticoagulant effects of the AT-heparin complex [Schoen P. et al. Thromb. Haemost. 66:435-441 (1991)]. As previously showed by some of the inventors, another highly specific heparinoid-binding protein found in platelets is heparanase that would serve to hydrolyze heparinoids, thereby abolishing their anticoagulant activities [Nasser (2006) ibid]. Degradation of heparinoids by heparanase, however, requires prolonged incubation times (in hours) under non-physiological conditions (acidic pH) [Nasser (2006) ibid], and therefore it is questionable whether this would be the only way heparanase could counteract heparinoid activities.

In clinical use for over 50 years, heparin is an important and widely used anticoagulant for the prophylaxis or treatment of thromboembolic disease as well as numerous other applications, such as treatment and prophylaxis of Deep vein thrombosis (DVT) and pulmonary embolism (PE), Acute coronary syndromes, Percutaneous coronary intervention (PCI), Thromboembolic disorders, Arterial embolization, Vascular and cardiac surgery and Extracorporeal circulation (hemodialysis, hemofiltration, and cardiopulmonary bypass during cardiac surgery). Unfortunately, heparin can cause serious adverse events, such as uncontrolled bleeding or other conditions such as heparin-induced thrombocytopenia (HIT).

In select patient populations (e.g., cardiac surgery) exposed to heparin, up to 50% can develop heparin-dependent antibodies. Up to 5% of all patients exposed to heparin develop HIT. Thromboembolic complications have been reported to occur in half to two thirds of patients with HIT, including those with and without thrombosis at diagnosis. Clinical data have shown that approximately 20% of patients with thrombotic complications lose a limb, and about 30% die without appropriate alternative nonheparin therapy.

In the present invention, the effect of heparanase precursor on coagulation functions, predominantly under physiological conditions, was examined. The present invention show for the first time that the nonactive proenzyme form of heparanase as well as a heparanase-derived heparin-binding peptide can rapidly reverse or counteract heparinoid-mediated anticoagulant activities.

As shown by the following examples, heparanase pro-enzyme has no effects on the major coagulation activities. However, heparanase pro-enzyme has profound inhibitory effects on heparinoids-mediated regulation of coagulation responses, apparently via mechanisms that are not enzymatic. Moreover, the present invention further shows that a peptide comprising the amino acid sequence of heparin-binding domain within heparanase converts the anticoagulating effect of heparinoids. Therefore, heparanase pro-enzyme and peptides thereof, may act as a pro-coagulant factor promoting clot formation in the presence of heparinoids.

The following observations support the newly discovered nonenzymatic role(s) of heparanase in haemostasis, as presented by the invention: (i) The enzyme utilized in this study was in its inactive, proenzyme state that is stable at physiological pH. (ii) Platelet heparanase was found to degrade endothelial cell HS at pH 6.0 but not at pH 7.4, even though 25% of maximum activity was detected at pH 7.4 [Yahalom J. et al. J. Clin. Invest. 74:1842-1849 (1984); Ihrcke N. S. et al. J. Cell Physiol. 175:255-267 (1998)]. However, inactivation of heparanase at pH 7.4 did not affect heparin binding [Yahalom (1984) ibid.; Ihrcke (1998) ibid]. (iii) Heparanase concentrations of less then 5 μg/ml had no pro-coagulant effects (not shown). Hence, the active pro-coagulant concentrations of heparanase were stoichiometric (μg/ml range) with respect to heparin, indicative for a heparin-sequestering activity of heparanase. (iv) The heparin-neutralizing activity of heparanase proenzyme was quite rapid and much faster than expected for the degradation of heparin, the latter even under optimal conditions. (v) Active platelet-derived heparanase reduced unfractionated heparin to about the size of LMWH [Gong F. J. Biol. Chem. 278:35152-35158 (2003)], which is still highly active as cofactor for FXa inhibition by AT. In agreement with this notion, a previous study showed that heparanase cleaves target structures on heparinoids that are distinct from the AT-binding sequence [Gong (2003) ibid.]. (vi) As shown by the present invention, the heparanase-derived peptide also prevented the FXa inhibition cofactor activity of heparinoids. Clearly, this assay did not include any enzymatic part of heparanase.

The contribution of heparanase in the regulation of local haemostatic responses should take into account the physiological context of these processes. Since heparanase is released from platelets upon activation, the active enzyme would act on exposed subendothelial basement membrane at sites of blood vessel injury and thereby accelerate its disintegration or destruction, a process in contradiction with the onset of primary haemostasis. Thus, without being bound by any theory, based on the data of the present invention and the mentioned information, the inventors propose that heparanase function during haemostasis is independent of its enzymatic activity and expressed as heparinoid-sequestering activity to stabilize the phase of clot formation, in accordance with previous considerations described above. Further proof for the procoagulant nature of heparanase proenzyme comes from data of heparanase transgenenic mice, whose plasmatic coagulation functions were elevated, based on e.g. a shortened clotting time in aPTT assay [Nasser (2006) ibid].

More particularly, the present invention demonstrated that enzymatically-inactive heparanase can reverse the anti-coagulant effects of unfractionated as well as LMWH. Moreover, the reversal of FXa inhibition by plasma derived from LMWH-treated patients indicated that various forms of heparanase (or peptides derived thereof) may act as antidots for heparinoids in clinical settings, including LMWH treated patients. Since low molecular weight species of heparinoids are favorable for clinical usage due to their high efficacy and improved pharmacokinetics [Wong G. C. JAMA 289:331-342 (2003)], heparanase could provide a new endogenous and safe antagonistic principle by which the clinical usage of LMWH would be controlled. Although other heparinoid-counteracting substances, including protamine sulfate or platelet factor 4, have been used with variable success, they are not effective in reversing LMWH activity [Massonnet-Castel S. Et al. Haemostasis 16:139-146 (1986)]. These substances can cause haemodynamic changes and other clinical complications [Kanbak M. et al. Anaesth. Intensive Care 24:559-563 (1996); Kimmel S. E. et al. Anesth Analg 94:1402-1408 (2002)], or may be clinically problematic due to the involvement of platelet factor 4 in heparin-induced thrombocytopenia pathogenesis [Rauova L. et al. Blood 107:2346-2353 (2006)]. It should be further noted that the highly active bacterial heparanase I enzyme has been previously tested in patients for its heparin neutralizing potential, but shown to be not equivalent to protamine because of its inferior safety profile [Stafford-Smith M. et al. Anesthesiology 103:229-240 (2005)].

More specifically, the pro-coagulant effects of heparanase pro-enzyme discovered by the present invention, may be utilized to reverse the clinical effects of anticoagulants in the absence of proper anti-dots or may help to counteract bleeding complications and any other condition related to the anticoagulating activity of heparinoids.

Thus, in a first aspect, the present invention relates to a composition for the inhibition of heparinoids anti-coagulation activity. The composition of the invention comprises as an active ingredient a eukaryotic endoglycosidase or any mutant, fragment or peptide thereof comprising at least one heparin-binding domain. This composition may optionally further comprise a pharmaceutically acceptable carrier, diluent excipient and/or additive.

According to one embodiment, the present invention relates to a composition for the inhibition of heparinoids anti-coagulation activity in a subject in need thereof. The composition of the invention comprises as an active ingredient a eukaryotic endoglycosidase or any mutant, fragment or peptide thereof comprising at least one heparin-binding domain. This composition may optionally further comprise a pharmaceutically acceptable carrier, diluent excipient and/or additive.

In a specifically preferred embodiment, the invention provides a pharmaceutical composition for the treatment and prevention of a coagulation related pathologic clinical condition. Such composition specifically comprises as an active ingredient an eukaryotic endoglycosidase or any mutant, fragment or peptide thereof comprising at least one heparin-binding domain, said composition optionally further comprising a pharmaceutically acceptable carrier, diluent excipient and/or additive.

As indicated above, the composition of the invention is intended for inhibiting heparinoids anticoagulating activity. As used herein in the specification and in the claims section below, the term “inhibit” and its derivatives refers to suppress or restrain from free expression of activity. According to a preferred embodiment of the present invention at least about 20-90%, preferably, at least about, 40-90%, more preferably, at least about 80-90% of the heparinoid anti-coagulating activity is abolished by the non-active heparanase or the peptides used by the invention.

According to one preferred embodiment, the eukaryotic endoglycosidase comprised as an active ingredient within the composition of the invention, may be a non-active endoglycosidase or any mutant, fragment or peptide thereof comprising at least one heparin-binding domain.

More specifically, the non-active endoglycosidase may preferably be the 65 Kd latent form of mammalian heparanase pro-enzyme. Alternatively, the non-active endoglycosidase may be a mutated heparanase molecule or any variant, fragment or peptide thereof comprising at least one heparin-binding domain, devoid of heparanase endoglycosidase catalytic activity.

As used herein in the specification and in the claims section below, the phrase “heparanase catalytic activity” or its equivalent “heparanase activity” refer to an animal endoglycosidase hydrolyzing activity which is specific for heparin or heparan sulfate proteoglycan substrates, as opposed to the activity of bacterial enzymes (heparinase I, II and III) which degrade heparin or heparan sulfate by means of β-elimination.

By “functional fragments” is meant “fragments”, “variants”, “analogs” or “derivatives” of the molecule. A “fragment” of a molecule, such as any of the amino acid sequence of the 65 kDa non-active heparanase or any mutants thereof used by the present invention is meant to refer to any amino acid subset of the molecule, including at least one heparin-binding domain. A “variant” of such molecule is meant to refer to a naturally occurring molecule substantially similar to either the entire molecule or a fragment thereof. An “analog” of a molecule is a homologous molecule from the same species or from different species. By “functional” is meant having same biological function, for example, required for reversing the anti coagulating activity of heparinoids.

According to another preferred embodiment, the heparin-binding domain comprised within the endoglycosidase or any mutant, fragment or peptide thereof may comprise the amino acid sequence of any one of residues Lys¹⁵⁸-Asp¹⁷¹, Gln²⁷⁰-Lys²⁸⁰ and Lys⁴¹¹-Arg⁴³² of mammalian heparanase. It should be noted that according to a particular embodiment, the mammalian heparanase may preferably be the human heparanase. Therefore, the preferred heparin-binding domains are located in three regions of human heparanase. One region comprises amino acid residues Lys¹⁵⁸ to Asp¹⁷¹ (also denoted as SEQ ID NO:1), the second region comprises amino acid residues Lys262 to Lys²⁸⁰ (also denoted as SEQ ID NO:2), and the third region comprises amino acid residues Lys⁴¹¹ to Arg⁴³² (also denoted as SEQ ID NO:3), or any functionally equivalent fragment, derivative, and variant thereof. Example for derivative is the Lys¹⁵⁸ to Asp¹⁷¹ with an additional cysteine as denoted by SEQ ID NO: 4.

It should be appreciated that as used herein in the specification and in the claim section below, all the amino acid locations (Lys¹⁵⁸ to Asp¹⁷¹, Lys²⁶² to Lys²⁸⁰ and Lys⁴¹¹ to Arg⁴³²) refer to the position of the amino acid sequence of human heparanase as denoted by GenBank Accession No. AF144325.

In one particular embodiment, the present invention thus provides a composition comprising as active agent at least one peptide as defined in the invention. Thus, said composition shall comprise as active agent a peptide comprising an amino acid sequence of heparin-binding site within heparanase, specifically a peptide comprising any one of Lys¹⁵⁸ to Asp¹⁷¹, Lys²⁶² to Lys²⁸⁰, Lys⁴¹¹ to Arg⁴³² , and any functionally equivalent fragments or derivatives thereof.

According to another specifically preferred embodiment, a preferred active ingredient comprised within the composition of the invention may be a peptide, preferably, about 1 to 40 amino acid long, more preferably, about 5 to 20 amino acids and most preferably, about 10 to 20 amino acid residues long, comprising the amino acid sequence of any one of residues Lys¹⁵⁸-Asp¹⁷¹, residues Gln²⁷⁰-Lys²⁸⁰ and residues Lys⁴¹¹-Arg⁴³² of human heparanase (also denoted by SEQ ID NO: 1, 2, 3 and 4, respectively). Preferably, the peptide comprises the amino acid sequence as denoted by any one of SEQ ID NO: 1, 2, 3, 4 or any analogs and derivatives thereof. More preferably, the peptide comprises the amino acid sequence as denoted by SEQ ID NO: 1 or any fragments, analogs and derivatives thereof, such as the peptide of SEQ ID NO: 4.

The terms analogs and derivatives as used herein mean peptides comprising the 1 to 40 amino acid residues, more preferably, about 5 to 20 amino acids and most preferably, about 10 to 20 amino acid residues, of the amino acid sequence of any one of SEQ ID NO: 1, SEQ ID NO:2, SEQ ID NO: 3 and SEQ ID NO:4, with any insertions, deletions, substitutions and modifications to the peptide that do not interfere with the ability of said peptide to inhibit heparnoids anti coagulating activity (hereafter referred to as “derivative/s”). A derivative should maintain a minimal homology to said amino acid sequence, e.g. between 20 to 90%, preferably, between 40 to 75%, more preferably, even less than 30%. It should be appreciated that by the term “insertions” as used herein is meant any addition of amino acid residues to the peptides of the invention, between 1 to 50 amino acid residues, preferably, between 20 to 1 amino acid residues and most preferably, between 1 to 10 amino acid residues.

Further, the peptides used by the invention may be extended at the N-terminus and/or C-terminus thereof with various identical or different amino acid residues. As an example for such extension, the peptide may be extended at the N-terminus and/or C-terminus thereof with identical or different hydrophobic amino acid residue/s which may be naturally occurring or synthetic amino acid residue/s. One example for a synthetic amino acid residue is D-alanine.

An additional and preferred example for such an extension may be provided by peptides extended both at the N-terminus and/or C-terminus thereof with a cysteine residue. Naturally, such an extension may lead to a constrained conformation due to Cys-Cys cyclization resulting from the formation of a disulfide bond.

Another example may be the incorporation of an N-terminal lysyl-palmitoyl tail, the lysine serving as linker and the palmitic acid as a hydrophobic anchor.

In addition, the peptides used as an active ingredient of the composition of the invention may be extended by aromatic amino acid residue/s, which may be naturally occurring or synthetic amino acid residue/s. A preferred aromatic amino acid residue may be tryptophan. Alternatively, the peptides can be extended at the N-terminus and/or C-terminus thereof with amino acids present in corresponding positions of the amino acid sequence of the naturally occurring heparanase.

Nonetheless, according to the invention, the peptides of the invention may be extended at the N-terminus and/or C-terminus thereof with various identical or different organic moieties which are not naturally occurring or synthetic amino acids. As an example for such extension, the peptide may be extended at the N-terminus and/or C-terminus thereof with an N-acetyl group.

The lack of structure of linear peptides renders them vulnerable to proteases in human serum and acts to reduce their affinity for target sites, because only few of the possible conformations may be active. Therefore, it is desirable to optimize the peptide structure, for example by creating different derivatives of the various peptides of the invention.

In order to improve peptide structure, the peptides of the invention can be coupled through their N-terminus to a lauryl-cysteine (LC) residue and/or through their C-terminus to a cysteine (C) residue, or to other residue/s suitable for linking the peptide to adjuvant/s for immunization, as will be described in more detail hereafter.

It should be noted that the peptides used by the invention, as well as derivatives thereof may all be positively charged, negatively charged or neutral and may be in the form of a dimer, a multimer or in a constrained conformation. A constrained conformation can be attained by internal bridges, short-range cyclizations, extension or other chemical modification.

For every single peptide sequence used by the invention and disclosed herein, this invention includes the corresponding retro-inverso sequence wherein the direction of the peptide chain has been inverted and wherein all the amino acids belong to the D-series.

It is to be appreciated that the present invention also includes longer peptides in which part or all of the basic Lys¹⁵⁸ to Asp¹⁷¹ amino acid residues (as denoted by SEQ ID NO: 1) which are comprised in the amino acid sequence. Longer peptides may also be a result of a tandem repetition, in which the basic peptidic sequence (of the 1 to 40 amino acid long, more preferably, about 5 to 20 amino acids and most preferably, about 10 to 20 amino acid residues long peptide used by the invention) is repeated from about 2 to about 100 times.

Interestingly, it should be noted that it was recently shown by the present inventors (WO 2005/071070), that these peptides and particularly, the fourteen amino acid peptide of SEQ ID NO: 1, exhibit high affinity and physically interact with immobilized heparin and HS. Moreover, these peptides were able to significantly inhibit heparanase enzymatic activity, by interfering with heparanase catalytic activity, and are thus also used as heparanase inhibitors for the treatment of diseases and disorders caused by or associated with heparanase catalytic activity such as cancer, inflammatory disorders, autoimmune diseases or kidney disorders. This effect is most likely attributed to inhibition of the interaction between heparanase and the heparin/HS substrate. NMR studies recently performed by the present inventors clearly identified Lys¹⁵⁸, Lys¹⁵⁹ and Lys¹⁶¹ as important mediators of heparanase-heparin/HS interaction. Therefore, it should be appreciated that these residues may be particularly relevant for the inhibition of heparinoids anti-coagulating activity by non-active heparanase or peptides thereof.

In yet another embodiment, the composition of the invention is intended for the inhibition of the anti-coagulating activity of heparinoids such as heparin, low molecular weight heparin (LMWH), unfractionated heparin (UFH), or any functional fragment and derivatives thereof.

It should be noted that the invention further encompasses inhibition of heparinoids or “heparin-like molecules”. The term heparin-like molecule as used herein refers to a molecule that possesses anti-coagulant activity and chemical structure sufficiently similar to that of heparin such that said molecule is considered as a possible alternate therapy to a patient requiring heparin. A heparin-like molecule includes, but is not limited to, a low molecular weight heparin, a heparin analogue, and the like. The term low molecular weight heparin includes heparin molecules having a molecular weight of less than 8,000 daltons. The term heparin analogue comprises heparinoids, such as hepramine and its salts, chondroitins and their salts, and the like. The term heparin as used herein refers to standard commercially available heparin and derivatives thereof. The term standard heparin encompasses a mixture of unfractionated heparin molecules having an average molecular weight of between about 8,000 and about 30,000 daltons or any subfraction thereof. In addition, it is contemplated that the term heparin as used herein encompasses biologically active heparin molecules that are isolated from a mammalian source, that are chemically modified, or that are partially or completely synthesized de novo. The term heparin derivative encompasses salts of heparin, heparin fragments and the like.

Heparin administration is the standard antithrombotic therapy indicated for acute venous thrombosis, for prophylaxis of thrombosis in the post-surgical (especially orthopedic) and immobile patient, and for flushing of intravenous lines to maintain patency. However, due to their potency, heparin and LMWH suffer drawbacks. Uncontrolled bleeding as a result of the simple stresses of motion and accompanying contacts with physical objects or at surgical sites is the major complication. In addition, approximately 5% (range up to 30%) of patients treated with heparin, and about 2% of patients receiving unfractionated heparin (UFH), develop immune-mediated thrombocytopenia (HIT) which may be complicated by either bleeding (as a consequence of decreased platelet count) or by arterial and venous thrombosis due to intravascular platelet clumping. This complication occurs in as many as 20% of patients with HIT and may result in serious morbidity and death in about 50% of the cases. Treatment with heparin in the case of HIT results in serious aggravation of the hemostatic complications, hence, heparin therapy in that case should be discontinued. On the other hand, discontinuation of heparin may expose the patient, who requires anti-thrombotic therapy, to excessive risk of thrombosis since no alternate therapy with immediate and effective antithrombotic capacity is presently available. Moreover, the alternate therapies, such as low-molecular weight heparin or heparinoids, may not be compatible because of potential crossreaction with the anti-heparin antibodies, resulting in further aggrevation of HIT. Heparin-induced thrombocytopenia (HIT) may be complicated in 30-75% of cases by a paradoxical thrombotic syndrome (HITTS), either arterial or venous. HITTS carries relevant rates of mortality and morbidity, amongst which are cerebral and/or myocardial infarction and limb amputations. It is unclear as yet why some patients suffer from isolated thrombocytopenia (HIT), whilst others have HITTS.

Heparin-Induced Thrombocytopenia (HIT) is therefore a life-threatening immune disorder. A diagnosis of HIT is considered when patients develop unexplained thrombocytopenia and/or thromboembolic complications in association with recent heparin therapy. Thrombocytopenia and thrombosis typically occur 5-10 days after treatment has been initiated in naive individuals, but complications develop sooner in those with prior drug exposure. Platelet counts typically range between 20,000/μL and 100,000/μL at presentation. However, the diagnosis should be considered in any exposed individual whose platelet count falls by 30-50% in the absence of another clearly identified cause. Unlike most other causes of drug-induced or immune-mediated thrombocytopenia, bleeding is not commonly seen in HIT. Rather, 30-50% of patients with HIT paradoxically develop thrombosis, a complication referred to as Heparin-Induced Thrombocytopenia and Thrombosis (HITT). Patients with HITT can develop venous or arterial thrombi. Venous thromboembolic complications occur more commonly than arterial thrombosis. Patients with thrombocytopenia as their only manifestation of HIT, also referred to as isolated HIT, often have unrecognized venous thrombi. Arterial thrombi are common in vessels traumatized by catheterization or surgery, but stroke, myocardial infarction and peripheral gangrene have all been reported.

HIT, which usually develops after a patient has been on heparin for 5 or more days, may develop sooner if there has been previous heparin exposure. Heparin binds to platelet factor 4 (PF4), forming a highly reactive antigenic complex on the surface of platelets and on endothelial cell surfaces, thereby increasing the number of targets for heparin-dependent antibodies. Susceptible patients then develop an antibody (IgG) to the heparin-PF4 antigenic complex. Once produced, immunoglobulins, usually IgG, bind to the heparin-PF4 immune complex on the platelet surface. The Fc portion of the IgG then activates the platelets by binding to platelet Fc receptors. Thrombocytopenia develops as the reticuloendothelial system consumes activated platelets, platelet microaggregates, and IgG-coated platelets. Most devastating, however, is the thrombotic state that develops as a result of platelet activation and the generation of procoagulant microparticles, and an additional increase in thrombin generation.

HIT is therefore a serious side effect of a drug that is widely used in clinical practice. All patients exposed to heparin, administered by any route or at any dose, are at varying risk of developing HIT and its potentially devastating thrombotic complications. This includes patients receiving UFH at full therapeutic doses and low prophylactic doses, including the minute amounts in heparin flushes and on heparin-coated catheters. Patients receiving LMWH are also at risk for HIT, although to a lower degree. With 12 million patients receiving either UFH or LMWH in the United States each year, the clinical implications of HIT become readily apparent.

Clearly, low molecular species of heparinoids are favorable for clinical usage due to their highly effectiveness and improved pharmacokinetics [Bussey H, et al., 24(8 Pt 2):103S-107S (2004)]. However, one of the imperative difficulties of LMWH clinical usage is the absence of an adequate anti-dot. Moreover, Protamine sulfate which is utilized as the current anti-dot for UFH can not reverse effectively LMWH activity, and can cause hemodynamic changes and other serious side effects [Chawla L S, et al., Obes. Surg. May; 14(5):695-8 (2004); Mixon T A, Semin Thromb Hemost. 30(3):369-77 (2004)]. The new direct thrombin inhibitors also have no specific anti-dot [Warkentin T E, Can. J. Anaesth. 49(6):S11-25 (2002)].

Therefore, in another preferred embodiment, the composition of the invention is particularly intended for the inhibition of heparinoids anti-coagulation activity in a subject in need thereof, preferably, a mammalian subject suffering of a coagulation-related pathologic clinical condition. More specifically, such clinical condition may be a condition related to or caused by the anti-coagulating effect of heparinoids, for example, uncontrolled bleeding or immune-mediated thrombocytopenia (HIT).

It should be further noted that inhibition of the ant-coagulant activity of heparinoids may be also desired (as a preoperative or post operative treatment) in patients in need of a surgical intervention. Particularly, patients receiving heparinoids as a regular treatment.

The composition of the invention may comprise the active substance in free form and be administered directly to the subject to be treated. Alternatively, depending on the size of the active molecule, it may be desirable to conjugate it to a carrier prior to administration. Therapeutic formulations may be administered in any conventional dosage formulation. Formulations typically comprise at least one active ingredient, as defined above, together with one or more acceptable carriers thereof.

Each carrier should be both pharmaceutically and physiologically acceptable in the sense of being compatible with the other ingredients and not injurious to the patient. Formulations include those suitable for oral, rectal, nasal, or parenteral (including subcutaneous, intramuscular, intraperitoneal, intravenous and intradermal) administration. The formulations may conveniently be presented in unit dosage form and may be prepared by any methods well known in the art of pharmacy. The nature, availability and sources, and the administration of all such compounds including the effective amounts necessary to produce desirable effects in a subject are well known in the art and need not be further described herein.

The pharmaceutical forms suitable for injection use include sterile aqueous solutions or dispersions and sterile powders for the extemporaneous preparation of sterile injectable solutions or dispersions. In all cases the form must be sterile and must be fluid to the extent that easy syringeability exists. It must be stable under the conditions of manufacture and storage and must be preserved against the contaminating action of microorganisms, such as bacteria and fungi.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above.

In the case of sterile powders for the preparation of the sterile injectable solutions, the preferred method of preparation are vacuum-drying and freeze drying techniques which yield a powder of the active ingredient plus any additional desired ingredient from a previously sterile-filtered solution thereof.

It should be noted that these are applicable for all composition described by the present invention.

More specifically, said non-catalytic 65 Kd heparanase, mutations, fragments or peptides thereof, or any substance or a composition comprising the same having heparinoids inhibitory activity, may be administered by the methods of the invention, systemically, for example by parenteral, e.g. intravenous, intraperitoneal or intramuscular injection. In another example, the pharmaceutical composition can be introduced to a site by any suitable route including intravenous, subcutaneous, transcutaneous, topical, intramuscular, intraarticular, subconjunctival, or mucosal, e.g. oral, intranasal, or intraocular administration. Still further, the compositions of the invention may be administered by a route selected from parenteral, intravaginal, intranasal, mucosal, sublingual and rectal administration and any combinations thereof.

Local administration to the area in need of treatment may be achieved by, for example, local infusion during surgery, topical application, and direct injection into the desired location.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

As indicated above, pharmaceutical compositions for use in accordance with the present invention may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the invention may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

Pharmaceutical compositions for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

For oral administration, the pharmaceutical preparation may be in liquid form, for example, solutions, syrups or suspensions, or in solid form as tablets, capsules and the like. For administration by inhalation, the compositions are conveniently delivered in the form of drops or aerosol sprays. For administration by injection, the formulations may be presented in unit dosage form, e.g. in ampoules or in multidose containers with an added preservative.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions, and the like, for oral ingestion by a patient.

Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol, cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). Thickeners, flavoring agents, diluents, emulsifiers, dispersing aids or binders may be desirable.

If desired, disintegrating agents may be added, such as cross-linked polyvinyl pyrrolidone, agar, or alginic acid or a salt thereof such as sodium alginate.

Accordingly, the invention further encompasses an eukaryotic endoglycosidase or any mutant, fragment or peptide thereof comprising at least one heparin-binding domain dosage forms that can be intraorally administered. The terms “intraoral administration” and “intraorally administering” include administration by adsorption through any surface inside the mouth or upper throat (such as the cheek (e.g., the inner cheek lining), gums, palate, tongue, tonsils, periodontal tissue, lips, and the mucosa of the mouth and pharynx). These terms, for example, include sublingual and buccal administration.

The administration compositions may alternately be in the form of a solid, such as a tablet, capsule or particle, such as a powder or sachet. Solid dosage forms may be prepared by manually or physically blending the solid form of the delivery agent compound with the solid form of an eukaryotic endoglycosidase or any mutant, fragment or peptide thereof comprising at least one heparin-binding domain.

For administration by nasal inhalation, the active ingredient for use according to the present invention, an eukaryotic endoglycosidase or any mutant, fragment or peptide thereof comprising at least one heparin-binding domain, may conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The preparations described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of the present invention may also be formulated in rectal compositions such as suppositories or retention enemas, using, e.g., conventional suppository bases such as cocoa butter or other glycerides.

Thus, the pharmaceutical compositions of the present invention include, but are not limited to, solutions emulsions, and liposome-containing formulations. These compositions may be generated from a variety of components that include, but are not limited to, preformed liquids, self-emulsifying solids and self-emulsifying semisolids.

Pharmaceutical compositions suitable for use in context of the present invention include compositions wherein the active ingredients are contained in an amount effective to achieve the intended purpose. More specifically, a therapeutically effective amount means an amount of active ingredients effective to prevent, alleviate or ameliorate symptoms of disease or prolong the survival of the subject being treated.

Determination of a therapeutically effective amount is well within the capability of those-skilled in the art.

The amount of the therapeutic or pharmaceutical composition of the invention which is effective in the treatment of a particular disease, condition or disorder will depend on the nature of the disease, condition or disorder and can be determined by standard clinical techniques. In addition, in vitro assays as well in vivo experiments may optionally be employed to help identify optimal dosage ranges. The precise dose to be employed in the formulation will also depend on the route of administration, and the seriousness of the disease, condition or disorder, and should be decided according to the judgment of the practitioner and each patient's circumstances. Effective doses may be extrapolated from dose-response curves derived from in vitro or animal model test systems.

As used herein, “effective amount” means an amount necessary to achieve a selected result. For example, an effective amount of the composition of the invention useful for inhibition of the anti coagulating activity of heparinoids and thereby for the treatment of said pathology.

In a particular embodiment preferred effective amount of pro-heparanase enzyme may range between 0.1 μg to 0.1 mg/ml, preferably, between 1 to 10 μg/ml. In another preferred embodiment, where the peptide, preferably, the peptide of SEQ ID NO: 1, is used by the invention, preferred effective amount of peptide may range between 100 to 0.001 mg/ml, preferably, between 1 to 0.01 mg/ml.

In a second aspect, the invention relates to a method for the inhibition of heparinoids anti-coagulation activity in a subject in need thereof. The method according to the invention comprises the step of administering to said subject an inhibitory effective amount of a eukaryotic endoglycosidase or any mutant, fragment or peptide thereof comprising at least one heparin-binding domain or of any composition comprising the same.

The present invention further provides for a method for the treatment and/or prevention of a subject suffering from a coagulation-related pathologic clinical condition, comprising the step of administering to said subject an inhibitory effective amount of a eukaryotic endoglycosidase or any mutant, fragment or peptide thereof comprising at least one heparin-binding domain or of any composition comprising the same.

According to one preferred embodiment of said aspect, the eukaryotic endoglycosidase used by the methods of the invention may be a non-active endoglycosidase or any mutant, fragment or peptide thereof comprising at least one heparin-binding domain. More specifically, such heparin-binding domain comprises the amino acid sequence of any one of residues Lys¹⁵⁸-Asp¹⁷¹, Gln²⁷⁰-Lys²⁸⁰ and Lys⁴¹¹-Arg⁴³² of mammalian heparanase.

According to a specifically preferred embodiment, the non-active endoglycosidase used by the method of the invention may be the 65 Kd latent form of mammalian heparanase pro-enzyme or alternatively, a non-active endoglycosidase may be a mutated heparanase molecule devoid of heparanase endoglycosidase catalytic activity.

According to another specifically preferred embodiment, the methods of the invention use a peptide comprising the amino acid sequence of any one of residues Lys¹⁵⁸-Asp¹⁷¹, Gln²⁷⁰-Lys²⁸⁰ and Lys⁴¹¹-Arg⁴³² of mammalian heparanase, for the inhibition of heparinoids anti-coagulating activity.

In yet another specifically preferred embodiment, the methods of the invention use a peptide which comprises the amino acid sequence as denoted in any one of SEQ ID NO: 1, 2, 3, 4, preferably, the peptide of SEQ ID NO: 1, or any analogs and derivatives thereof, for the inhibition of heparinoids anti-coagulating activity in a subject in need thereof. A non-limiting example for such derivative is the peptide of SEQ ID NO: 4.

According to another preferred embodiments, the heparinoid inhibited by the methods of the invention may be heparin, low molecular weight heparin (LMWH), unfractionated heparin (UFH), or any functional fragment thereof.

According to another specifically preferred embodiment, the methods of the invention are intended for the treatment of a mammalian subject suffering of a coagulation-related pathologic clinical condition, preferably, a condition related to or caused by the anti-coagulating effect of heparinoids. More specifically, such condition may be uncontrolled bleeding. It should be noted that the method of the invention may be applicable also for the treatment of other complications related to the use of heparinoids, such as immune-mediated thrombocytopenia (HIT).

According to a particular embodiment, the method of the invention may also be useful for prevention of a coagulation-related pathologic clinical condition. This may be particularly applicable in surgical conditions, specifically of subjects which are treated with heparinoids, in order to avoid, reduce or prevent bleeding. Therefore, according to this embodiment, the method comprises preoperative and/or postoperative administration of a therapeutically effective amount of a eukaryotic endoglycosidase or any mutant, fragment or peptide thereof comprising at least one heparin-binding domain or of any composition comprising the same, to a subject in need of a surgical intervention. It should be further noted that the composition of the invention may be applied topically or in any other suitable route of administration.

It should be further noted that the compositions and methods of the invention may be also applicable for preventing and reducing bleeding from any injury, that is, any injured tissue in living organisms. The injured tissue may be an intra-corporeal tissue, such as an inside wall of a stomach, a fracture, or the like, a skin surface or the like, and also a soft tissue, such as a spleen, or a hard tissue, such as bone. The injury may be a lesion, trauma or wound, or one formed by an infection or from a surgical operation.

The pharmaceutical composition used by the method of the invention can be prepared in dosage units forms and may be prepared by any of the methods well-known in the art of pharmacy. In addition, the pharmaceutical composition may further comprise pharmaceutically acceptable additives such as pharmaceutical acceptable carrier, excipient or stabilizer, and optionally other therapeutic constituents. Naturally, the acceptable carriers, excipients or stabilizers are non-toxic to recipients at the dosages and concentrations employed.

The magnitude of therapeutic dose of the composition of the invention will of course vary with the group of patients (age, sex, etc.), the nature of the condition to be treated and with the route administration, all of which shall be determined by the attending physician.

Although the method of the invention is particularly intended for the treatment of pathologic clinical conditions associated with the anti-coagulating activity of heparinoids in humans, other mammals are included.

“Treatment” refers to therapeutic treatment. Those in need of treatment are mammalian subjects suffering from any coagulation-related pathologic disorder. By “patient” or “subject in need” is meant any mammal for which administration of an eukaryotic endoglycosidase or any mutant, fragment or peptide thereof comprising at least one heparin-binding domain, or any pharmaceutical composition comprising this compound or derivatives thereof is desired, in order to prevent, overcome or slow down such infliction. “Mammal” or “mammalian” for purposes of treatment refers to any animal classified as a mammal including, human, research animals, domestic and farm animals, and zoo, sports, or pet animals, such as dogs, horses, cats, cows, etc. In a particular embodiment said mammalian subject is a human subject.

To provide a “preventive treatment” or “prophylactic treatment” is acting in a protective manner, to defend against or prevent something, especially a condition or disease.

The method of the invention should be applied to a subject suffering from a coagulation-related disorder, particularly caused by heparinoids. As used herein, the term “pathologic condition” refers to a condition in which there is a disturbance of normal functioning. Such condition is any abnormal condition of the body or mind that causes discomfort, dysfunction, or distress to the person affected or those in contact with the person. Sometimes the term is used broadly to include injuries, disabilities, syndromes, symptoms, deviant behaviors, and atypical variations of structure and function, while in other contexts these may be considered distinguishable categories. It should be noted that the terms “disease”, “disorder”, “condition” and “illness”, are equally used herein.

The invention further provides a method for the inhibition of heparinoids anti-coagulation activity. This method comprises the step of: (a) contacting an inhibitory effective amount of an eukaryotic endoglycosidase or any mutant, fragment or peptide thereof comprising at least one heparin-binding domain or of any composition comprising the same, with said heparinoid under suitable conditions creating a mixture;

(b) adding to the mixture obtained in step (a), a mammalian body fluid sample, preferably plasma, under suitable conditions for a suitable time; and (c) examining the anticoagulation activity of said heparinoids on said sample, as compared to a suitable control, by a suitable means.

It should be appreciated that this method as described by the invention may be also used for monitoring a treated patient and/or for determining the precise amount of an eukaryotic endoglycosidase or any mutant, fragment or peptide thereof needed, in any time point of the treatment, for successful inhibition of heparinoids anti-coagulation activity in a treated subject in need. It should be further noted that this method enables a real-time monitoring of the desired concentration needed to be administered to the subject in need.

Examination of the effect on coagulation may be performed using different well established coagulation assays, as also performed by the present invention. One example for such suitable assay may be the prothrombin time (PT) test which measures how long it takes for a clot to form in a sample of blood. In the body, the clotting process involves a series of sequential chemical reactions. One of the final steps is the conversion of prothrombin to thrombin. Prothrombin is one of several clotting factors that are produced by the liver. The PT test evaluates the integrated function of these factors and the body's ability to produce a clot in a reasonable amount of time.

Another preferred assay may be the Activated Partial Thromboplastin Time (aPPT). This test is a useful and effective method for screening patients with a bleeding tendency, for evaluating the effect of therapy in procoagulant disorders and as the basis for several specific coagulant factor assay procedures. The aPTT has been widely used as a test for monitoring and regulating heparin therapy.

Another test may be the Thrombin Time (TT) assay which reflects the time taken for a plasma sample to clot on addition of thrombin. The test is essentially a measure of fibrinogen to fibrin conversion and the factors affecting this terminal stage of the coagulation system.

Antithrombin (previously known as Antithrombin III) is an important natural anticoagulant. Its function is to inhibit the activities of various serine proteinase enzymes produced during the clotting process. This includes not only thrombin as its name suggests, but also FXa, FIXa, IXa and probably FVIIa.

Antithrombin acts as a relatively inefficient coagulation inhibitor on its own. Its inhibitory activity is greatly (about 5,000-fold) accelerated by heparin. In fact, heparin's anticoagulant activity is almost entirely mediated via Antithrombin, and patients with Antithrombin deficiency are relatively resistant to heparin anticoagulation. Therefore, as a further coagulation test, examining the reversing effect of the composition of the invention, may be the measurement of Antithrombin activity in the patient's plasma (AT). This may be a chromogenic assay for Antithrombin in which a fixed excess amount of purified thrombin is added to the test sample. After incubation in the presence of heparin, residual (non-inactivated) thrombin is measured with a specific chromogenic substrate. The normal range is 83-115%.

It should be noted that other coagulation assays may be used, for example, the Protein S and Protein C activity test. Protein S (PS) is one of the vitamin K-dependent coagulation proteins and is synthesized in the liver as an inactive precursor. The active form is obtained after carboxylation of glutamic residues by a vitamin K-dependent carboxylase, thus allowing the molecule to bind calcium ions. Unlike the other clotting factors in this family, however, PS is not a zymogen of a serine proteinase. The PS Activity assay is based upon the cofactor activity of PS which is enhances the anticoagulant action of activated Protein C. This enhancement is reflected by the prolongation of the clotting time of a system enriched with Factor Va which is a physiological substrate for activated Protein C. Protein C is a member of the Vitamin K-dependent coagulation factor family. Unlike its procoagulant relatives, Factors II, VII, IX and X, Protein C acts as a natural anticoagulant by downregulating thrombin generation after coagulation has been initiated. Protein C is activated by thrombin bound to thrombomodulin on the endothelial cell surface. Activated Protein C (APC) then combines with its cofactor, Protein S, on the surface of the platelet where it can degrade and inactivate factor Va and factor VIIIa. In the absence of Protein C, thrombin generation goes relatively unchecked and a hypercoagulable state ensues.

It should be noted that platelet-aggregation test using various mediators may also be used for evaluating the pro-coagulation effect of the non-active heparanase and peptides thereof used by the invention.

According to one embodiment, the eukaryotic endoglycosidase used by the method of the invention may be a non-active endoglycosidase or any mutant, fragment or peptide thereof comprising at least one heparin-binding domain.

According to a specifically preferred embodiment, the non-active endoglycosidase may preferably be the 65 Kd latent form of mammalian heparanase pro-enzyme, or alternatively, may be a mutated heparanase molecule devoid of heparanase endoglycosidase catalytic activity.

It should be noted that the non-active 65 Kd form of heparanase, or any fragments thereof used by all methods and compositions of the invention may be provided as a purified recombinant heparanase protein, a fusion heparanase protein, a nucleic acid construct encoding the non-active 65 Kd form heparanase, a host cell expressing said construct, a cell, a cell line and a tissue endogenously expressing the non-active 65 Kd form of heparanase, or any lysates thereof. It should be also appreciated that were a peptide is used by the methods and compositions of the invention, such peptide may be produces synthetically, purified and isolated in any procedure known in the art. Alternatively, such peptide may be produced recombinantly.

According to another preferred embodiment, the method of the invention may use a non-active endoglycosidase or any mutant, fragment or peptide thereof comprising at least one heparin-binding domain. Such domain may preferably comprises the amino acid sequence of any one of residues Lys¹⁵⁸-Asp¹⁷¹, residues Gln²⁷⁰-Lys²⁸⁰ and residues Lys⁴¹¹-Arg⁴³² (also denoted by SEQ ID NOs. 1, 2, and 3, respectively) of human heparanase. It should be noted that all the amino acid locations (Lys¹⁵⁸ to Asp¹⁷¹, Lys²⁶² to Lys²⁸⁰ and Lys⁴¹¹ to Arg⁴³²) refer to the amino acid sequence of human heparanase as denoted by GenBank Accession No. AF144325.

According to one specifically preferred embodiment, the method of the invention may use a peptide comprising the amino acid sequence of any one of residues Lys¹⁵⁸-Asp¹⁷¹ residues Gln²⁷⁰-Lys²⁸⁰ and residues Lys⁴¹¹-Arg⁴³², of human heparanase. Preferably, the method of the invention uses a peptide comprising the amino acid sequence as denoted by SEQ ID NO: 1 or any analogs and derivatives thereof. In one example, the SEQ of ID NO: 4, is a derivative of SEQ ID NO: 1.

According to another specifically preferred embodiment, the method of the invention is intended for the inhibition of the anti-coagulation activity of different heparinoids such as heparin, low molecular weight heparin (LMWH), unfractionated heparin (UFH), or any functional fragment thereof.

According to a third aspect, the present invention relates to the use of a eukaryotic endoglycosidase or any mutant, fragment or peptide thereof comprising at least one heparin-binding domain, in the preparation of a composition for the inhibition of heparinoids anti-coagulation activity.

Still further, the invention relates to the use of a eukaryotic endoglycosidase or any mutant, fragment or peptide thereof comprising at least one heparin-binding domain, in the preparation of a composition for the inhibition of heparinoids anti-coagulation activity in a subject in need thereof.

The invention further provides the use of a eukaryotic endoglycosidase or any mutant, fragment or peptide thereof comprising at least one heparin-binding domain, in the preparation of a composition for the treatment and prevention of a coagulation related pathologic clinical condition.

According to one preferred embodiment, the eukaryotic endoglycosidase used by the invention may be a non-active endoglycosidase or any mutant, fragment or peptide thereof comprising at least one heparin-binding domain. Such heparin-binding domains may comprise the amino acid sequence of any one of residues Lys¹⁵⁸-Asp¹⁷¹, Gln²⁷⁰-Lys²⁸⁰ and Lys⁴¹¹-Arg⁴³² of mammalian heparanase.

More specifically, the non-active endoglycosidase may preferably be the 65 Kd latent form of mammalian heparanase pro-enzyme, or alternatively, a mutated heparanase molecule devoid of heparanase endoglycosidase catalytic activity.

According to another preferred embodiment, the invention uses a peptide comprising the amino acid sequence of any one of residues Lys¹⁵⁸-Asp¹⁷¹, Gln²⁷⁰-Lys²⁸⁰ and Lys⁴¹¹-Arg⁴³² of mammalian heparanase. Preferably, a peptide comprising the amino acid sequence as denoted by SEQ ID NO: 1 or any analogs and derivatives thereof, for the inhibition of heparinoids anti-coagulating activity. According to a preferred embodiment, heparinoid may be heparin, low molecular weight heparin (LMWH), unfractionated heparin (UFH), or any functional fragment thereof.

According to another preferred embodiment, of the invention uses the non-active forms of heparanase or of peptides thereof for inhibiting the anticoagulating activity of heparinoids and for the treatment of a subject in need thereof. Preferably, mammalian subject suffering of a coagulation-related pathologic clinical condition. Such coagulation-related pathologic clinical condition may be a disorder related to or caused by the anti-coagulating effect of heparinoids. For example, uncontrolled bleeding or immune-mediated thrombocytopenia (HIT).

It should be noted that the invention also encompasses the use of the heparanase pro-enzyme or peptides thereof as a preventive preoperative or post operative treatment of patients, particularly those receiving regularly heparinoids, when such patients undergo any surgical intervention, in order to prevent bleeding.

It should be appreciated that the invention further provides a method for making a medicament for the treatment of a coagulation-related pathologic condition caused by the anti-coagulating effect of heparinoids. Accordingly, the method of the invention comprises the step of: (a) providing a therapeutically effective amount of an eukaryotic endoglycosidase or any mutant, fragment or peptide thereof comprising at least one heparin-binding domain; (b) admixing said non-active form of pro-heparanase with at least one of a pharmaceutically acceptable carrier, diluent, excipient and/or additive.

Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

Throughout this specification and the Examples and claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

Experimental Procedures

Human Recombinant Non-Active 65 Kd Heparanase

The latent 65 kDa heparanase protein was purified from the conditioned medium of Chinese hamster ovary (CHO) cells, stably expressing the human heparanase gene construct in the mammalian pSecTag vector (Invitrogen) containing Myc and His tags at the protein C-terminus. The cells were grown in DMEM supplemented with 10% FCS, glutamine, pyruvate and antibiotics. For heparanase purification, the cells were grown over night in serum free-DMEM and the conditioned medium (˜1 liter) was purified on a Fractogel EMD SO₃⁻ (MERCK) column. The bound material was eluted with 1M NaCl and was further purified by affinity chromatography on anti-Myc tag antibody (Santa Cruz Biotechnology) column. We obtained at least 95% pure heparanase preparation by this two-step procedure.

Peptides

Peptides were synthesized on an ABIMED AMS 422 multiple peptide synthesizer (Langenfeld, Germany), employing the N-(9-fluorenyl) methoxycarbonyl (Fmoc) strategy following the commercial protocols. Peptide chains assembly was conducted on a 2-chlorotrityl chloride resin (Novabiochem). Crude peptides were purified to homogeneity by reverse-phase high pressure liquid chromatography on a semi-preparative silica C-18 column (250×10 mm; Lichrosorb RP-18, Merck). Elution was accomplished by a linear gradient established between 0.1% trifluoroacetic acid in water and 0.1% trifluoroacetic acid in 70% acetonitrile in water (v/v). The compositions of the products were determined by amino acid analysis (Dionex automatic amino acid analyzer, Sunnyvale, Calif.) following exhaustive acid hydrolysis. Molecular weights were ascertained by mass spectrometry (VG Tofspec; Laser Desorption Mass Spectrometry; Fison Instruments, Manchester, UK).

Heparin UFH and LMWH

Commercial porcine intestine heparin (UFH) was obtained from Kamada Ltd (Beit Kama, Israel). Low-molecular-weight heparin (Enoxaparin) was purchased from Aventis (Strasbourg).

Coagulation Assays
aPTT-Automatic determination of activated partial thromboplastin time (aPTT) of samples was performed in duplicates, by mixing pooled normal plasma with aPTT reagent (FSL actin, Dade Behring). After exactly 10 min, the clotting time was determined on Sysmex CA1500 analyzer.
Xa activity—Photometric determination of anti-Xa activity was performed to evaluate the activity of LMWH in human plasma on Sysmex CA1500 analyzer, using the LMWH Kit (Chromogenics).
PT—Prothrombin time (PT) was determined using the Innovin reagent (Dade Behring) on Sysmex CA1500 analyzer.
Protein C—Protein C activity was determined by the Berichrom Protein C kit, and ATIII was measured by the chromogenic Berichrom Antithrombin III kit (both from Dade Behring).
Platelet aggregation—Platelet aggregation was measured on normal donor platelets utilizing the Payton Aggregocorder type aggregometer (Payton Associates).
Protein S—Free Protein S was measured with the Liatest kit (Diagnostica Stago). All coagulation assays were subjected to external quality control program (NEQUAS, Sheffield, UK).

### Example 1

Heparanase Pro-Enzyme has No Direct Effect on Coagulation Functions

To examine the possible involvement of heparanase in the coagulation process, variety of coagulation tests including activated partial thrombin time (aPTT, testing the intrinsic coagulation pathway), prothrombin time (PT, testing the extrinsic coagulation pathway), thrombin time (TT, testing thrombin mediated fibrin generation), as well as protein C and protein S (both coagulation inhibitors) were performed by the inventors.

The effects of heparanase on platelet aggregation stimulated by a variety of mediators (e.g. ADP, collagen, thrombin), was next tested. As shown in Table 1, all of these coagulation functions were not affected by the presence of heparanase pro-enzyme, and were within the normal ranges.

TABLE 1
Heparanase pro-enzyme does not affect
directly coagulation functions

| Coagulation assay, | Heparanase | Normal |
|---|---|---|
| factor determination | (10 μg/ml) * | range/value |
| Prothrombin time (PT) | 10.6 | 10.03-12.43 (sec) |
| Activated partial pro- | 29.4 | 25-34 (sec) |
| thrombin time (aPTT) | | |
| Thrombin time (TT) | 17.3 | 14-21 (sec) |
| Protein C-activity | 111 | 75-151% |
| Protein S-activity | 105 | 54-138% |
| Antithrombin III | 103 | 74-114% |
| Factor Xa-activity | 102 | 100% |

| | |
|---|---|
| Platelet aggregation | No effects of heparanase |

*A single representative experiment out of 2-4 performed is shown.

### Example 2

Heparanase Pro-Enzyme Reverses the Heparin-Induced Reduction in aPTT and TT Responses

The extent of blood coagulation responses requires the balance by anticoagulant components in the microenvironment of the endothelium, represented by cell surface HSPG and associated coagulation inhibitors. Heparanase, which is released from platelets upon activation may function as a physiological procoagulant. Therefore, the inventors tested the effects of heparanase on heparinoid-mediated down-regulation of coagulation activities, under conditions which do not support its enzymatic activities (e.g. the usage of the inactive heparanase proenzyme, under neutral pH). Two coagulation assays are affected by heparinoids: activated partial thromboplastin time (aPTT) which measures the intrinsic coagulation pathway, and thrombin time (TT) which measures the thrombin-mediated conversion of fibrinogen to fibrin. As illustrated by FIG. 1, in both cases, heparin forms a ternary complex with the natural thrombin inhibitor antithrombin III (ATIII) and thrombin, resulting in thrombin inactivation. Heparin significantly reduces both the aPTT as well as the TT response. The inventors therefore next examined the effect of heparanase on the heparin induced reduction of both, aPTT and TT responses. As clearly demonstrated by FIGS. 2 and 3, in the presence of heparanase pro-enzyme, these responses are significantly reversed, specifically, at low heparin concentrations. As expected, neither heparin nor heparanase pro-enzyme affected the PT response, indicating specificity of the heparin/heparanase regulatory mode within the intrinsic coagulation pathway (not shown). Apparently, the complex formed between heparin and ATIII necessary to augment the inhibition of thrombin and thus affect fibrin generation as demonstrated by the results shown in FIG. 2, and illustrated by the scheme of FIG. 1. This complex was significantly interfered in the presence of heparanase pro-enzyme, as shown by FIG. 3. The heparin-induced prolongation of the thrombin time (measuring thrombin activity) was significantly prevented by heparanase pro-enzyme, thus partially destroying the anti-thrombin effects of heparin.

### Example 3

Heparanase Pro-Enzyme Reverses the Anti-Coagulant Effect of Heparin by Restoring Factor Xa Activity in vitro and in Plasma of Human Patients Treated with LMWH

As illustrated by FIG. 1, an additional mode of ATIII activity is the formation of an inhibitory complex with activated coagulation Factor X (Xa). This factor associates with factor Va and prothrombin to form the prothrombinase complex on the endothelium, leading to thrombin generation and subsequent clot formation. Unfractionated or low molecular weight heparinoids (LMWH) bind to AT, and induce conformational changes resulting in binding and inhibition of Factor Xa activity.

In recent years LMWH became widely used anti-coagulants due to their prominent anti-coagulant activity, and improved pharmacokinetics compared with un-fractionated heparin. However, there is still no existing anti-dot to inhibit these clinically highly abundant anti-coagulants, in the case of urgent clinical needs. Such needs may occur during extensive bleedings (e.g. brain) caused by LMWH overdose, or un-controlled bleedings in LMWH treated individuals due to other medical causes. Therefore, the effects of heparanase pro-enzyme on the anti-coagulant activities of heparinoids, was next tested by the inventors. As expected, low molecular weight heparin reduces significantly Factor Xa activity (FIG. 4). However, while heparanase proenzyme had no effect on FXa activity alone (Table 1), in the presence of AT, increasing doses of heparanase proenzyme reversed the heparin inhibitory effects, restoring FXa activity to the normal level (FIG. 4). Similar effects of heparanase were observed on plasma treated with unfractionated heparin (not shown).

In order to assess possible clinical effects of heparanase pro-enzyme as an anti-dot to LMWH, heparanase pro-enzyme effect on Factor Xa activity ex vivo, was next tested in plasma derived from patients treated with LMWH, which were incubated with 10 •g/ml heparanase proenzyme. As shown by FIG. 5, heparanase pro-enzyme elevated significantly Factor Xa activity in samples obtained form twelve different LMWH-treated patient, in a dose dependent manner. Thus, heparanase pro-enzyme reduced FXa inhibitory effects of LMWH in individual patient plasma samples, even at high levels of LMWH. Overall, the level of FXa inhibitory activity of LMWH in treated patients was reduced approximately by 50% (FIG. 5B).

### Example 4

Heparanase Derived Peptide, Comprising the Heparin-Binding Domain of Residues Lys158-Asp171, Completely Reverses the Factor Xa Inhibitory Effect of Heparin

It should be emphasized that the results using heparanase pro-enzyme, which is a non-active form of heparanase, clearly indicate that pro-coagulant activities of heparanase can not be attributed to its enzymatic activity. Without being bound by any theory, the inventors therefore speculate that enzymatically inactive heparanase may still bind heparinoids, and neutralize their anti-coagulant activities by their sequestering and/or competition with plasma resident anti-coagulants (e.g. ATIII). Three potential heparin-binding domains were identified, and one of them is mapped at the N terminus of the 50-kDa active heparanase subunit. A peptide corresponding to this region (Lys158-Asp171, also denoted by SEQ ID NO: 1) physically associates with heparin and heparan sulfate. Moreover, as previously shown by the inventors (WO 2005/071070) this particular peptide inhibited heparanase enzymatic activity in a dose-responsive manner, presumably through competition with the heparan sulfate substrate. Therefore, the inventors next tested whether the Lys158-Asp171 (SEQ ID NO. 1) heparin-binding peptide can abolish the Factor Xa inhibitory effects of heparin. As shown in FIG. 6, the Lys158-Asp171 heparin-binding peptide (as also denoted by SEQ ID NO: 1) completely reverse the Factor Xa inhibitory effect of heparin, as well as of LMWH and UFH (FIG. 7), albeit at a relatively high concentration (1 mg/ml), while the control, scrambled peptide had no effect.

These pro-coagulant effects of heparanase pro-enzyme may be utilized to reverse the clinical effects of anticoagulants in the absence of proper anti-dots (e.g. in the case on LMWH) or may help to counteract bleeding complications.

1-38. (canceled)

39. A composition for the inhibition of heparinoids anti-coagulation activity, comprising as an active ingredient an eukaryotic endoglycosidase or any mutant, fragment or peptide thereof comprising at least one heparin-binding domain, said composition optionally further comprising a pharmaceutically acceptable carrier, diluent excipient and/or additive.

40. The composition according to claim 39, for the inhibition of heparinoids anti-coagulation activity in a subject in need thereof.

41. The composition according to claim 40, for the treatment and prevention of a coagulation related pathologic clinical condition, wherein said composition comprises as an active ingredient an eukaryotic endoglycosidase or any mutant, fragment or peptide thereof comprising at least one heparin-binding domain, said composition optionally further comprising a pharmaceutically acceptable carrier, diluent excipient and/or additive.

42. The composition according to claim 39, wherein said eukaryotic endoglycosidase is a non-active endoglycosidase or any mutant, fragment or peptide thereof comprising at least one heparin-binding domain and wherein said non-active endoglycosidase is any one of the 65 Kd latent form of mammalian heparanase pro-enzyme and a mutated heparanase molecule devoid of heparanase endoglycosidase catalytic activity.

43. he composition according to claims 42, wherein said heparin-binding domain comprises the amino acid sequence of any one of residues Lys¹⁵⁸-Asp¹⁷¹, Gln²⁷⁰-Lys²⁸⁰ and Lys⁴¹¹-Arg⁴³² of mammalian heparanase.

44. The composition according to claim 43, wherein said peptide is a peptide comprising the amino acid sequence of any one of residues Lys¹⁵⁸-Asp¹⁷¹ as denoted by SEQ ID NO. 1, residues Gln²⁷⁰-Lys²⁸⁰ as denoted by SEQ ID NO. 2, residues Lys⁴¹¹-Arg⁴³² as denoted SEQ ID NO. 3 of human heparanase and a peptide comprising the amino acid sequence as denoted by SEQ ID NO: 4 or any fragments analogs and derivatives thereof.

45. The composition according to claim 39, wherein said heparinoid is any one of heparin, low molecular weight heparin (LMWH) and unfractionated heparin (UFH), and any functional fragment thereof.

46. The composition according to claims 40, wherein said subject in need is a mammalian subject suffering of a coagulation-related pathologic clinical condition, and wherein said condition is related to or caused by the anti-coagulating effect of heparinoids.

47. The composition according to claim 46, wherein said condition is any one of uncontrolled bleeding, immune-mediated thrombocytopenia (HIT) and a preoperative or postoperative condition.

48. A method for the inhibition of heparinoids anti-coagulation activity in a subject in need thereof comprising the step of administering to said subject an inhibitory effective amount of a eukaryotic endoglycosidase or any mutant, fragment or peptide thereof comprising at least one heparin-binding domain or of any composition comprising the same, wherein said eukaryotic endoglycosidase is a non-active endoglycosidase or any mutant, fragment or peptide thereof comprising at least one heparin-binding domain, and wherein said non-active endoglycosidase is any one of the 65 Kd latent form of mammalian heparanase pro-enzyme and a mutated heparanase molecule devoid of heparanase endoglycosidase catalytic activity.

49. A method for the treatment and prevention of a coagulation related pathologic clinical condition, comprising the step of administering to a subject in need thereof an inhibitory effective amount of a eukaryotic endoglycosidase or any mutant, fragment or peptide thereof comprising at least one heparin-binding domain or of any composition comprising the same.

50. The method according to claim 49, wherein said eukaryotic endoglycosidase is a non-active endoglycosidase or any mutant, fragment or peptide thereof comprising at least one heparin-binding domain, and wherein said non-active endoglycosidase is any one of the 65 Kd latent form of mammalian heparanase pro-enzyme and a mutated heparanase molecule devoid of heparanase endoglycosidase catalytic activity.

51. The method according to claims 50, wherein said heparin-binding domain comprises the amino acid sequence of any one of residues Lys¹⁵⁸-Asp¹⁷¹, residues Gln²⁷⁰-Lys²⁸⁰ and residues Lys⁴¹¹-Arg⁴³² of human heparanase.

52. The method according to claim 49, wherein said peptide is a peptide comprising the amino acid sequence of any one of residues Lys¹⁵⁸-Asp¹⁷¹ as denoted by SEQ ID NO. 1, residues Gln²⁷⁰-Lys²⁸⁰ as denoted by SEQ ID NO. 2, residues Lys⁴¹¹-Arg⁴³² as denoted by SEQ ID NO. 3 of human heparanase and a peptide comprising the amino acid sequence as denoted by SEQ ID NO: 4 or any fragments analogs and derivatives thereof.

53. The method according to claim 49, wherein said heparinoid is any one of heparin, low molecular weight heparin (LMWH) and unfractionated heparin (UFH), and any functional fragment thereof.

54. The method according to claim 49, wherein said subject in need is a mammalian subject suffering of a coagulation-related pathologic clinical condition, and wherein said coagulation related pathologic clinical condition is a condition related to or caused by the anti-coagulating effect of heparinoids.

55. The method according to claim 54, wherein said pathologic clinical condition is any one of uncontrolled bleeding and immune-mediated thrombocytopenia (HIT).

56. A method according to claim 49, wherein prevention of a coagulation-related pathologic clinical condition comprising preoperative and/or postoperative administration of a therapeutically effective amount of a eukaryotic endoglycosidase or any mutant, fragment or peptide thereof comprising at least one heparin-binding domain or of any composition comprising the same, to a subject in need of a surgical intervention.

57. A method for the inhibition of heparinoids anti-coagulation activity comprising the step of:
(a) contacting an inhibitory effective amount of a eukaryotic endoglycosidase or any mutant, fragment or peptide thereof comprising at least one heparin-binding domain or of any composition comprising the same with said heparinoid under suitable conditions creating a mixture;
(b) adding to the mixture obtained in step (a), a mammalian body fluid sample, preferably plasma, under suitable conditions for a suitable time;
(c) examining the anticoagulation activity of said heparinoids on said sample, as compared to a suitable control, by a suitable means.
